(19) United States
(12) Patent Application Publication
(10) Pub. No.: US 2016/0143843 A1
(43) Pub. Date: May 26, 2016

(54) SKIN CREAM

(71) Applicant: Masqline LLC, North Miami beach, FL (US)

(72) Inventor: Alan H. Steinberg, North Miami Beach, FL (US)

(21) Appl. No.: 14/918,875

(22) Filed: Oct. 21, 2015

Related U.S. Application Data
(60) Provisional application No. 62/067,146, filed on Oct. 22, 2014.

(51) Int. Cl. A61K 8/97; A61Q 19/00; A61K 8/34; A61K 8/66; A61K 8/36; A61K 8/49; A61K 8/41; A61K 8/19; A61K 8/63; A61K 8/37

(52) U.S. Cl. CPC A61K 8/97; A61K 8/63; A61Q 19/007; A61K 8/342; A61K 8/34; A61K 8/375; A61K 8/361; A61K 8/498; A61K 8/41; A61K 8/19; A61K 8/66; A61K 2800/805

(57) ABSTRACT

The present disclosure encompasses formulations, and methods of manufacture and use thereof, for topical application to and conditioning of human skin. The formulations disclosed comprise an effective amount of at least one steroidal agent, plant extract, viscosity-adjusting agent, anti-oxidant. In particular, the formulations comprise an effective amount micronized progesterone and are used for topical application to the skin of men.

### CROSS-REFERENCE TO RELATED APPLICATIONS

This application claims the benefit of U.S. Provisional Patent Application No. 62/067,146, filed Oct. 22, 2014, the contents of which are incorporated by reference herein in its entirety.

### FIELD OF INVENTION

The present invention relates to formulations, and methods of manufacture and use thereof, for topical application to and conditioning of human skin. The formulations comprise an effective amount of at least one steroidal agent, e.g., micronized progesterone, and are used for topical application to the skin of men.

### BACKGROUND

Historically, people have regularly used lotions and creams for medical, cosmetic, and therapeutic applications. Soft, supple and flexible skin has a marked cosmetic appeal and is an attribute of normal functioning epidermis. As human skin ages with advancing years, the epidermis can become folded, ridged or furrowed to form wrinkles, signaling loss of youthful appearance and heralding the transition to old age. Exposure to excessive doses of sunlight accelerates the transition process. In particular, the outer layer of the epidermis (the stratum corneum) can become dry and flaky following exposure to cold weather or excessive contact with detergents or solvents. A consequence of the resulting loss of skin moisture is the perception that the skin is losing its soft, supple and flexible characteristics.

Such aging and drying effects on the skin can be a source of irritation, pain and great concern for users. In an attempt to address these issues, the market has provided several types of non-prescription, over-the-counter, topical skin compositions to moisturize skin. While there are several products that moisturize the skin, their effectiveness on various conditions such as rosacea, fine lines and wrinkles, mild eczema and psoriasis, as well as their anti-aging skin conditioning effects are less than acceptable for many users. Moreover, these compositions have generally, if not almost exclusively, been formulated for use by women, rather than men.

Therefore, a need exists for topical formulations, as well as methods for their manufacture and use, that are effective for improving the perceived skin qualities of the subjects, particularly the male subjects, to which they are applied. In particular, there is a related need in that such formulations that do not include a compound or component that would require a prescription.

### BRIEF SUMMARY

The present disclosure a formulation that comprises water, an effective amount of a steroidal agent, an effective amount of a plant extract, an effective amount of a viscosity-adjusting agent, an effective amount of an antioxidant, and an effective amount of a chelator. The formulation of the disclosure is a topical composition for application to the skin, particularly for topical application to the skin of a human, and more specifically, for topical application to the skin of a human male.

In one embodiment, the steroidal agent of the formulation is micronized progesterone.

In another embodiment, the viscosity-adjusting agent of the formulation is an emollient, an emulsifier, a surfactant, or a thickener.

In a further embodiment, the plant extract of the formulation is aloe vera gel, Mexican wild yam root extract, sweet almond oil, panax ginseng root extract, saw palmetto extract, damiana aphrodisiaca extract, gotu kola extract, sage oil (e.g., sage Salvia essential oil), dong quai root extract, castor oil, or combinations thereof.

In one embodiment, the viscosity-adjusting agent of the formulation is cetearyl alcohol, ceteareth 20, a mixture of cetearyl alcohol and ceteareth 20, glyceryl stearate, stearic acid, a mixture of cetyl esters, or combinations of two or more thereof.

In another embodiment the formulation disclosed comprises a chelator, which can be ethylenediaminetetraacetic acid or a salt thereof. In one aspect of this embodiment, the chelator is the disodium salt of ethylenediaminetetraacetic acid.

In a further embodiment, the disclosed formulation comprises an antioxidant, which antioxidant can be D,L-tocopherol or a glucose/glucose oxidase/lactoperoxidase mixture combined with an substrate mixture.

In another embodiment, the disclosed formulation comprises, by weight, from about 2% to about 8% micronized progesterone.

In one embodiment, the disclosed formulation comprises a plant extract, which plant extract is aloe vera, formulated using a 200:1 powder (which may derived from a CO₂ extract) within a range of from about 0.05% to about 0.5% by weight of the formulation. Identification and use of other preparations of aloe vera (e.g. liquids or gels) which are functionally equivalent or substantially identical to the powders employed herein, are within the level of skill of the ordinary artisan and are encompassed by the present disclosure and claims. In another aspect of this embodiment, the disclosed formulation comprises a plant extract, which plant extract is the Mexican wild yam root extract formulated within a range of from about 1.5% to about 11% by weight of the formulation, panax ginseng root extract (1:3 50%) formulated within a range of from about 1.5% to about 7% by weight of the formulation, saw palmetto extract (1:3 50%) formulated within a range of from about 0.5% to about 5% by weight of the formulation, damiana aphrodisiaca liquid extract (1:3 50%) formulated within a range of from about 1.5% to about 4.5% by weight of the formulation, gotu kola extract (1:3 50%) formulated within a range of from about 0.5% to about 5.0% by weight of the formulation, sage oil (e.g., sage Salvia essential oil) formulated within a range of from about 0.05% to about 1.5% by weight of the formulation, dong quai root extract formulated within a range of from about 1.5% to about 5.5% by weight of the formulation, castor oil formulated within a range of from about 3% to about 6% by weight of the formulation, or a combination of two or more thereof. In other embodiments, the panax ginseng root extract (1:3 50%) is formulated within a range of from about 2.0% to about 6.0% by weight of the formulation, from about 2.5% to about 5% by weight of the formulation from about 3% to about 4% by weight of the formulation. In a specific embodiment, the panax ginseng root extract (1:3 50%) is formulated at a level of about 3.5% by weight of the formulation. Again, although components of the formulation may be recited as a specific preparation (e.g., extracts identified as: 1:3 50%), identification and use of other preparations of such materials which are functionally equivalent or substantially identical to the specific preparations disclosed, are within the level of skill of the ordinary artisan and are encompassed by the present disclosure and claims.

In another embodiment, the disclosed formulation comprises a viscosity-adjusting agent, which viscosity-adjusting agent is cetearyl alcohol formulated within a range of from about 1% to about 8% by weight of the formulation, ceteareth 20 formulated within a range of from about 1% to about 8% by weight of the formulation, glyceryl stearate formulated within a range of from about 2.5% to about 7.5% by weight of the formulation, stearic acid formulated within a range of from about 1% to about 3% by weight of the formulation, cetyl esters formulated within a range of from about 0.5% to about 1.5% by weight of the formulation, a mixture of cetearyl alcohol and ceteareth 20 formulated within a range of from about 1.25% to about 7% by weight of the formulation, or a combination of two or more thereof.

In another embodiment, the disclosed formulation comprises a chelator, which chelator can be disodium ethylenediaminetetraacetic acid, formulated within a range of from about 0.1% to about 0.3% by weight of the formulation.

In a further embodiment, the disclosed formulation comprises antioxidant, which can be D,L-tocopherol formulated within a range of from about 0.5% to about 2.5% by weight of the formulation. In another embodiment, the antioxidant is a mixture comprising an enzyme component (a combination of glucose oxidase (1500-2750 units/ml) and lactoperoxidase (1000-1800 units/ml) and a substrate component (comprising 45-55% glucose, 0.66 to 0.8% potassium iodide, and 0.42 to 0.50% sodium thiocyanate) with the mixture formulated with a range of from about 0.5% to about 1.5% by weight of the formulation. In one aspect of this embodiment, the formulation comprises both of these antioxidants.

In another embodiment, the formulations of the disclosure comprise from about 40% to about 60% water, by weight. That is, water is added in a quantity sufficient to 100% by weight for the formulation.

In one embodiment, a formulation of the present disclosure comprises by weight, about 4% micronized progesterone, about 3.5% Mexican wild yam root extract, about 2% cetearyl alcohol and ceteareth 20, about 5% glyceryl stearate, about 2% stearic acid, about 5% sweet almond oil, about 1.5% D,L-alpha tocopheryl acetate oil, and about 0.2% disodium ethylenediaminetetraacetic acid.

In another embodiment, a formulation of the present disclosure comprises by weight, about 4% micronized progesterone, about 3.5% Mexican wild yam root extract, about 2% cetearyl alcohol and ceteareth 20, about 5% glyceryl stearate, about 2% stearic acid, about 5% sweet almond oil, about 1.5% D,L-alpha tocopheryl acetate oil, about 0.2% disodium ethylenediaminetetraacetic acid, about 51% water, and about 0.25% aloe vera 200:1 powder.

In another embodiment, a formulation of the present disclosure comprises by weight, about 4% micronized progesterone, about 3.5% Mexican wild yam root extract, about 2% cetearyl alcohol and ceteareth 20, about 5% glyceryl stearate, about 2% stearic acid, about 5% sweet almond oil, about 1.5% D,L-alpha tocopheryl acetate oil, about 0.2% disodium ethylenediaminetetraacetic acid, about 49% water, about 0.05% to about 0.5% aloe vera 200:1 powder, about 3.5% panax ginseng root extract (1:3 50%), about 3.5% saw palmetto extract (1:3 50%), about 3.5% damiana aphrodisiaca liquid extract (1:3 50%), about 2% gotu kola (1:3 50%), and about 0.2% sage extract (sage Salvia essential oil).

In a further embodiment, a formulation of the present disclosure comprises by weight, about 49.3% water, about 4% micronized progesterone, about 2% disodium ethylenediaminetetraacetic acid, about 5% cetearyl alcohol, about 4% castor oil, about 2% cetearyl alcohol and ceteareth 20, about 5% glyceryl monostearate, about 1.5% D,L-alpha tocopheryl acetate oil (96%), about 5% sweet almond extract, about 2% stearic acid, about 1% cetyl esters, about 0.25% aloe vera 200:1 powder; about 3.5% Mexican wild yam root liquid extract, about 3.5% panax ginseng root liquid extract (1:3 50%), about 3.5% saw palmetto liquid extract (1:3 50%), about 3.5% damiana aphrodisiaca liquid extract (1:3 50%), about 3.5% dong quai root liquid extract (1:3 50%), about 2% gotu kola liquid extract (1:3 50%), about 1% substrate (about 50% glucose, about 0.73% potassium iodide, and about 0.47% sodium thiocyanate), about 0.05% enzyme (lactoperoxidase 1000-1800 units/ml and glucose oxidase 1500-2750 units/ml), and about 0.2% sage Salvia essential oil.

In other embodiments, formulations of the present disclosure may further include an effective amount of a sunscreen component (“sunscreen agent”), with the amounts of the other components proportionately adjusted to accommodate the amount of sunscreen agent included in the formulation.

In one aspect of these embodiments, the formulation is prepared as a cream intended for topical application to the skin of a man.

In another embodiment, the present disclosure provides a method of conditioning the skin of a subject in need thereof, the method comprising topically applying an effective amount of the a formulation of the disclosure to the skin of the subject. In one aspect of this embodiment, the subject is a human male.

The present disclosure further provides a method of use of the formulations described, in which a small quantity of the formulation, e.g., 1 to 100 ml, is applied to exposed areas of the skin, from a suitable container or applicator and, if necessary, and then spread over and/or rubbed into the skin of the human subject, using the hand or fingers or a suitable device. In one aspect of this embodiment, the human subject is a male.

In another embodiment, the disclosed formulations are prepared as a lotion, a cream or a gel. The formulations can be packaged in a suitable container to suit its viscosity and intended use by the subject.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### DETAILED DESCRIPTION

The following description of the preferred embodiment(s) is merely exemplary in nature and is in no way intended to limit the invention, its application, or uses.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices and materials are now described. All publications mentioned herein are incorporated by reference for the purpose of describing and disclosing the materials and methodologies that are reported in the publication, which might be used in connection with the invention.

The present disclosure may be understood more readily by reference to the following detailed description of illustrative and preferred embodiments. It is to be understood that the scope of the claims is not limited to the specific ingredients, methods, conditions, devices, or parameters described herein, and that the terminology used herein is not intended to be limiting of the claimed invention. Also, as used in the specification, including the appended claims, the singular forms “a,” “an,” and “the” include the plural, and reference to a particular numerical value includes at least that particular value, unless the context clearly dictates otherwise. When a range of values is expressed, another embodiment includes from the one particular value and/or to the other particular value. Similarly, when values are expressed as approximations, by use of the antecedent basis “about,” it will be understood that the particular values forms another embodiment. All ranges are inclusive and combinable.

In certain embodiments, the term “about” indicates that the present disclosure contemplates that the specified value may be varied by ±20%. In other embodiments, the term “about” indicates that the present disclosure contemplates that the specified value may be varied by ±10%, ±5%, ±2%, or ±1%, while in other certain embodiments, the present disclosure contemplates that the specified value can be limited to its absolute numerical value, per se.

In certain embodiments, the formulations disclosed may comprise the combination of materials described herein, while in other embodiments, the formulations disclosed may consist essentially of materials described herein, or may consist of the materials described herein.

The phrase “effective amount,” as used herein, means an amount of a component or formulation sufficient to significantly induce a positive benefit, appearance, or feel to the skin of the subject to whom the formulation is applied, but low enough to avoid serious side effects, i.e., to provide a reasonable benefit to risk ratio, within the scope of sound judgment of the skilled artisan and/or end user.

The phrase “topical application,” as used herein means to apply or spread the formulations of the present disclosure onto the surface of the skin of a human subject.

The present disclosure provides formulations for topical application to the skin of a human. In particularly the formulations disclosed herein comprise about 4% micronized progesterone and are for topical application to the skin of men.

The present disclosure a formulation that comprises water, an effective amount of a steroidal agent, an effective amount of a plant extract, an effective amount of a viscosity-adjusting agent, an effective amount of an antioxidant, and an effective amount of a chelator. The formulation of the disclosure is a topical composition for application to the skin, particularly for topical application to the skin of a human, and more specifically, for topical application to the skin of a human male. In particular, water is added, as necessary, in a quantity sufficient to 100% by weight for the formulation.

In one embodiment, the steroidal agent of the formulation is micronized progesterone.

In another embodiment, the disclosed formulation comprises, by weight, from about 2% to about 8% micronized progesterone, or from about 2.5% to about 7% micronized progesterone, or from about 3% to about 6% micronized progesterone, or from about 4% to about 5% of micronized progesterone, by weight of the formulation. In a specific aspect of this embodiment, the disclosed formulation comprises about 4% micronized progesterone by weight of the formulation. In another specific aspect of this embodiment, the disclosed formulation comprises 4% micronized progesterone by weight of the formulation

In a further embodiment, the plant extract of the formulation is aloe vera, Mexican wild yam root extract, sweet almond oil, panax ginseng root extract, saw palmetto extract, damiana aphrodisiaca extract, gotu kola extract, sage oil, dong quai root extract, castor oil, or combinations thereof.

In one embodiment, the disclosed formulation comprises a plant extract, which plant extract is aloe vera gel 200:1 powder formulated within a range of from about 0.05% to about 0.50% by weight of the formulation, Mexican wild yam root extract formulated within a range of from about 1.5% to about 11% by weight of the formulation, sweet almond oil formulated within a range of from about 2.5% to about 7.5% by weight of the formulation, panax ginseng root extract (1:3 50%) formulated within a range of from about 1.5% to about 7% by weight of the formulation, saw palmetto extract (1:3 50%) formulated within a range of from about 1.5% to about 4.5% by weight of the formulation, damiana aphrodisiaca liquid extract (1:3 50%) formulated within a range of from about 1.5% to about 4.5% by weight of the formulation, gotu kola liquid extract (1:3 50%) formulated within a range of from about 0.5% to about 3.5% by weight of the formulation, sage oil formulated within a range of from about 0.5% to about 1.5% by weight of the formulation, dong quai root extract formulated within a range of from about 1.5% to about 5.5% by weight of the formulation, castor oil formulated within a range of from about 3% to about 6% by weight of the formulation, or a combination of two or more thereof.

In one embodiment, the disclosed formulation comprises a plant extract, which plant extract is aloe vera 200:1 powder formulated within a range of from about 0.05% to about 5% by weight of the formulation, Mexican wild yam root extract formulated within a range of from about 2.0 to about 9% by weight of the formulation, sweet almond oil formulated within a range of from about 3% to about 7% by weight of the formulation, panax ginseng root extract (1:3 50%) formulated within a range of from about 2.5% to about 5% by weight of the formulation, saw palmetto extract (1:3 50%) formulated within a range of from about 3% to about 4% by weight of the formulation, damiana aphrodisiaca liquid extract (1:3 50%) formulated within a range of from about 3% to about 4% by weight of the formulation, gotu kola liquid extract (1:3 50%) formulated within a range of from about 1.0% to about 3.0% by weight of the formulation, sage oil (sage Salvia essential oil) formulated within a range of from about 0.1% to about 1.0% by weight of the formulation, dong quai root extract formulated within a range of from about 2% to about 4.5% by weight of the formulation, castor oil formulated within a range of from about 3.25% to about 5% by weight of the formulation, or a combination of two or more thereof.

In one embodiment, the disclosed formulation comprises a plant extract, which plant extract is aloe vera 200:1 powder formulated within a range of from about 0.1% to about 0.4% by weight of the formulation, Mexican wild yam root extract formulated within a range of from about 3.5% to about 7% by weight of the formulation, sweet almond oil formulated within a range of from about 4% to about 6% by weight of the formulation, panax ginseng root extract (1:3 50%) formulated within a range of from about 3% to about 4% by weight of the formulation, saw palmetto extract (1:3 50%) formulated within a range of from about 3% to about 4% by weight of the formulation, damiana aphrodisiaca liquid extract (1:3 50%) formulated within a range of from about 1.5% to about 4.5% by weight of the formulation, gotu kola liquid extract (1:3 50%) formulated within a range of from about 1.5% to about 2.5% by weight of the formulation, sage oil (sage Salvia essential oil) formulated within a range of from about 0.15% to about 0.25% by weight of the formulation, dong quai root extract formulated within a range of from about 3% to about 4% by weight of the formulation, castor oil formulated within a range of from about 3.5% to about 4.5% by weight of the formulation, or a combination of two or more thereof.

In one embodiment, the disclosed formulation comprises a plant extract, which plant extract is aloe vera 200:1 powder formulated at a concentration of 0.2% to about 0.3% by weight of the formulation, Mexican wild yam root extract formulated at a concentration of about 3.5% by weight of the formulation, sweet almond oil formulated at a concentration of about 5% by weight of the formulation, panax ginseng root extract formulated at a concentration of about 3.5% by weight of the formulation, saw palmetto extract (1:3 50%) formulated at a concentration of about 3.5% by weight of the formulation, damiana aphrodisiaca liquid extract (1:3 50%) formulated at a concentration of about 3.5% by weight of the formulation, gotu kola liquid extract (1:3 50%) formulated at a concentration of about 2% by weight of the formulation, sage oil (sage Salvia essential oil) formulated at a concentration of about 0.2% by weight of the formulation, dong quai root extract formulated at a concentration of about 3.5% by weight of the formulation, castor oil formulated at a concentration of about 4% by weight of the formulation, or a combination of two or more thereof. In another aspect, the disclosed formulation comprises a plant extract, which plant extract is aloe vera 200:1 powder formulated at a concentration of 0.25% by weight of the formulation. In a further aspect, the disclosed formulation comprises gotu kola liquid extract (1:3 50%) formulated at a concentration of about 2% by weight of the formulation.

In another embodiment, the viscosity-adjusting agent of the formulation is an emollient, an emulsifier, a surfactant, or a thickener.

In one embodiment, the viscosity-adjusting agent of the formulation is cetearyl alcohol, ceteareth 20, a mixture of cetearyl alcohol and ceteareth 20, glyceryl stearate, stearic acid, a mixture of cetyl esters, or combinations of two or more thereof.

In another embodiment, the disclosed formulation comprises a viscosity-adjusting agent, which viscosity-adjusting agent is cetearyl alcohol formulated within a range of from about 1% to about 8% by weight of the formulation, ceteareth 20 formulated within a range of from about 1% to about 8% by weight of the formulation, glyceryl stearate formulated within a range of from about 2.5% to about 7.5% by weight of the formulation, stearic acid formulated within a range of from about 1% to about 3% by weight of the formulation, cetyl esters formulated within a range of from about 0.5% to about 1.5% by weight of the formulation, a mixture of cetearyl alcohol and ceteareth 20 formulated within a range of from about 1.25% to about 7% by weight of the formulation, or a combination of two or more thereof.

In a further embodiment, the disclosed formulation comprises a viscosity-adjusting agent, which viscosity-adjusting agent is cetearyl alcohol formulated within a range of from about 1.5% to about 5% by weight of the formulation, ceteareth 20 formulated within a range of from about 1.5% to about 5% by weight of the formulation, glyceryl stearate formulated within a range of from about 3% to about 7% by weight of the formulation, stearic acid formulated within a range of from about 1.25% to about 2.75% by weight of the formulation, cetyl esters formulated within a range of from about 0.7% to about 1.4% by weight of the formulation, a mixture of cetearyl alcohol and ceteareth 20 formulated within a range of from about 1.75% to about 3% by weight of the formulation, or a combination of two or more thereof.

In a further embodiment, the disclosed formulation comprises a viscosity-adjusting agent, which viscosity-adjusting agent is cetearyl alcohol formulated within a range of from about 4% to about 6% by weight of the formulation, ceteareth 20 formulated within a range of from about 1% to about 3% by weight of the formulation, glyceryl stearate formulated within a range of from about 4% to about 6% by weight of the formulation, stearic acid formulated within a range of from about 1.5% to about 2.5% by weight of the formulation, cetyl esters formulated within a range of from about 0.8% to about 1.2% by weight of the formulation, a mixture of cetearyl alcohol and ceteareth 20 formulated within a range of from about 1% to about 3% by weight of the formulation, or a combination of two or more thereof.

In a further embodiment, the disclosed formulation comprises a viscosity-adjusting agent, which viscosity-adjusting agent is cetearyl alcohol formulated at a concentration of about 5% by weight of the formulation, ceteareth 20 formulated at a concentration of about 2% by weight of the formulation, glyceryl stearate formulated at a concentration of about 5% by weight of the formulation, stearic acid formulated at a concentration of about 2% by weight of the formulation, cetyl esters formulated at a concentration of about 1% by weight of the formulation, a mixture of cetearyl alcohol and ceteareth 20 formulated at a concentration of about 2% by weight of the formulation, or a combination of two or more thereof.

In another embodiment the formulation disclosed comprises a chelator, which can be ethylenediaminetetraacetic acid or a salt thereof. In one aspect of this embodiment, the chelator is the disodium salt of ethylenediaminetetraacetic acid.

In another embodiment, the disclosed formulation comprises a chelator, which chelator can be disodium ethylenediaminetetraacetic acid, formulated within a range of from about 0.1% to about 0.3% by weight of the formulation.

In another embodiment, the disclosed formulation comprises a chelator, which chelator can be disodium ethylenediaminetetraacetic acid, formulated within a range of from about 0.15% to about 0.25% by weight of the formulation.

In another embodiment, the disclosed formulation comprises a chelator, which chelator can be disodium ethylenediaminetetraacetic acid, formulated within a range of from about 0.18% to about 0.22% by weight of the formulation.

In another embodiment, the disclosed formulation comprises a chelator, which chelator can be disodium ethylenediaminetetraacetic acid, formulated at a concentration of about 0.2% by weight of the formulation.

In a further embodiment, the disclosed formulation comprises an antioxidant, which antioxidant can be D,L-tocopherol or a glucose/glucose oxidase/lactoperoxidase mixture combined with a substrate mixture.

In a further embodiment, the disclosed formulation comprises antioxidant, which can be D,L-tocopherol formulated within a range of from about 0.5% to about 2.5% by weight of the formulation. In another embodiment, the antioxidant is a mixture comprising an enzyme component (a combination of glucose oxidase (1500-2750 units/ml) and lactoperoxidase (1000-1800 units/ml) and a substrate component (comprising 45-55% glucose, 0.66 to 0.8% potassium iodide, and 0.42 to 0.50% sodium thiocyanate) with the mixture formulated with a range of from about 0.5% to about 1.5% by weight of the formulation. In one aspect of this embodiment, the formulation comprises both of these antioxidants.

In a further embodiment, the disclosed formulation comprises antioxidant, which can be D,L-tocopherol formulated within a range of from about 0.9% to about 2.1% by weight of the formulation. In another embodiment, the antioxidant is a mixture comprising an enzyme component (a combination of glucose oxidase (1500-2750 units/ml) and lactoperoxidase (1000-1800 units/ml) and a substrate component (comprising 45-55% glucose, 0.66 to 0.8% potassium iodide, and 0.42 to 0.50% sodium thiocyanate) with the mixture formulated with a range of from about 0.7% to about 1.3% by weight of the formulation. In one aspect of this embodiment, the formulation comprises both of these antioxidants.

In a further embodiment, the disclosed formulation comprises antioxidant, which can be D,L-tocopherol formulated within a range of from about 1.3% to about 1.7% by weight of the formulation. In another embodiment, the antioxidant is a mixture comprising an enzyme component (a combination of glucose oxidase (1500-2750 units/ml) and lactoperoxidase (1000-1800 units/ml) and a substrate component (comprising 45-55% glucose, 0.66 to 0.8% potassium iodide, and 0.42 to 0.50% sodium thiocyanate) with the mixture formulated with a range of from about 0.9% to about 1.2% by weight of the formulation. In one aspect of this embodiment, the formulation comprises both of these antioxidants.

In a further embodiment, the disclosed formulation comprises antioxidant, which can be D,L-tocopherol formulated at a concentration of about 1.5% by weight of the formulation. In another embodiment, the antioxidant is a mixture comprising an enzyme component (a combination of glucose oxidase (1500-2750 units/ml) and lactoperoxidase (1000-1800 units/ml) and a substrate component (comprising 45-55% glucose, 0.66 to 0.8% potassium iodide, and 0.42 to 0.50% sodium thiocyanate) with the mixture formulated at a concentration of about 1.05% by weight of the formulation, e.g., 0.05% of the enzyme component and 1.0% of the substrate component. In one aspect of this embodiment, the formulation comprises both of these antioxidants.

In another embodiment, the formulations of the disclosure comprise from about 40% to about 60% water, by weight. In other aspects of this embodiment, formulations of the disclosure comprise from about 45% to about 55% water. In other aspects of this embodiment, formulations of the disclosure comprise from about 47% to about 53% water. In still other aspects of this embodiment, formulations of the disclosure comprise about 49% to about 51% water. In a specific embodiment, formulations of the disclosure comprise about 50% water by total weight of the formulation. That is, water is added in a quantity sufficient to 100% by weight for the formulation.

In one embodiment, a formulation of the present disclosure comprises by weight, about 4% micronized progesterone, about 3.5% Mexican wild yam root extract, about 2% cetearyl alcohol and ceteareth 20, about 5% glyceryl stearate, about 2% stearic acid, about 5% sweet almond oil, about 1.5% D,L-alpha tocopheryl acetate oil, and about 0.2% disodium ethylenediaminetetraacetic acid.

In another embodiment, a formulation of the present disclosure comprises by weight, about 4% micronized progesterone, about 3.5% Mexican wild yam root extract, about 2% cetearyl alcohol and ceteareth 20, about 5% glyceryl stearate, about 2% stearic acid, about 5% sweet almond oil, about 1.5% D,L-alpha tocopheryl acetate oil, about 0.2% disodium ethylenediaminetetraacetic acid, about 51% water, and about 0.25% aloe vera 200:1 powder.

In another embodiment, a formulation of the present disclosure comprises by weight, about 4% micronized progesterone, about 3.5% Mexican wild yam root extract, about 2% cetearyl alcohol and ceteareth 20, about 5% glyceryl stearate, about 2% stearic acid, about 5% sweet almond oil, about 1.5% D,L-alpha tocopheryl acetate oil, about 0.2% disodium ethylenediaminetetraacetic acid, about 49% water, about 0.25% aloe vera 200:1 powder, about 3.5% panax ginseng root extract (1:3 50%), about 3.5% saw palmetto extract (1:3 50%), about 3.5% damiana aphrodisiaca liquid extract (1:3 50%), about 2% gotu kola liquid extract (1:3 50%), and about 0.2% sage extract (sage Salvia essential oil).

In a further embodiment, a formulation of the present disclosure comprises by weight, about 49.3% water, about 4% micronized progesterone, about 2% disodium ethylenediaminetetraacetic acid, about 5% cetearyl alcohol, about 4% castor oil, about 2% cetearyl alcohol and ceteareth 20, about 5% glyceryl monostearate, about 1.5% D,L-alpha tocopheryl acetate oil (96%), about 5% sweet almond extract, about 2% stearic acid, about 1% cetyl esters, about 0.25% aloe vera 200:1 powder; about 3.5% Mexican wild yam root liquid extract, about 3.5% panax ginseng root liquid extract (1:3 50%), about 3.5% saw palmetto liquid extract (1:3 50%), about 3.5% damiana aphrodisiaca liquid extract (1:3 50%), about 3.5% dong quai root liquid extract (1:3 50%), about 2.0% gotu kola liquid extract (1:3 50%), about 1% substrate (about 50% glucose, about 0.73% potassium iodide, and about 0.47% sodium thiocyanate), about 0.05% enzyme (lactoperoxidase 1000-1800 units/ml and glucose oxidase 1500-2750 units/ml), and about 0.2% sage Salvia essential oil.

In other embodiments, formulations of the present disclosure may further include an effective amount of a sunscreen component (“sunscreen agent”), with the amounts of the other components proportionately adjusted to accommodate the amount of sunscreen agent included in the formulation. In certain embodiments, the sunscreen or combination of sunscreens, provides protection to UVA radiation, or to UVB radiation, or to both UVA and UVB radiation. Suitable sunscreens include, e.g., solid organic UV-filters such as but not limited to, benzophenone-3 (i.e., oxybenzone), 2-Hydroxy-4-methoxyphenyl)-(2-hydroxyphenyl)methanone (i.e., dioxybenzone), 2-(2H-benzotriazol-2-yl)-4-methylphenol, also known as drometrizole trisiloxane (also known as MEXORYL XL), butylmethoxy dibenzoylmethane (“avobenzone”), 4-methyl benzilidene camphor (“4-MBC”), ethylhexyl triazone (available as UVINUL T-150 from BASF of Ludwigshafen, Germany), diethylamino hydroxybenzoyl hexyl benzoate (“DHHB”) available as UVINUL A Plus from BASF; and Bemotrizinol (available as TINOSORB S from BASF), Menthyl-2-aminobenzoate (“menthyl anthranilate”), 4-Aminobenzoic acid (“PABA”), hydroxy methylphenyl benzotriazole, and combinations thereof. In other aspects of this embodiment, the sunscreen may be oxybenzone, avobenzone, drometrizole trisiloxane, ethylhexyl triazone, DHHB, Bemotrizinol, oxybenzone and avobenzone. The sunscreen agent may be included in the formulation at a level, by dry weight, within a concentration range from about 0.5% to about 30%, such as from about 1% to about 20%, such as from about 1.5% to about 15%, such as from about 1.5% to about 10%, such as from about 2% to about 10%, e.g., from about 3% to about 9% by weight. In certain embodiments, the sunscreen agent may be present in a concentration from about 0.5% to about 9%.

In one aspect of these embodiments, the formulation is prepared as a cream intended for topical application to the skin of a man.

The present disclosure also provides a method of conditioning the skin of a subject in need thereof, the method comprising topically applying an effective amount of the a formulation of the disclosure to the skin of the subject. In one aspect of this embodiment, the subject is a human male.

Application of the methodology outlined above has identified the specific components noted above that have been combined to provide presently-disclosed formulations that provide significant benefits, including but not limited to the “skin conditioning effects,” noted below, to those to whom they are topically applied.

The effects provided by topical application to the skin of an effective amount of the formulations of the present disclosure include but are not limited to one or more of the following attributes: improved elasticity, maintenance of firm, soft, and clear skin; promotion of the renewal of healthy skin cells and providing a “luminous glow” to the skin; moisturization and soothing of dry, flaky skin; overall enhancement of the appearance of treated skin; calming of irritations and reduction in visible skin puffiness; improvement in the appearance of dark circles under eyes; attraction and retention of water in the skin thereby providing protection of the skin from dehydration and development of premature wrinkles

The present disclosure further provides a method of use of the formulations described, in which a small quantity of the formulation, e.g., 1 to 100 mL, is applied to exposed areas of the skin, from a suitable container or applicator and, if necessary, and then spread over and/or rubbed into the skin of the human subject, using the hand or fingers or a suitable device. In one aspect of this embodiment, the human subject is a male. In certain embodiments, the amount of the formulation applied is within the range of from about 0.1 mL to about 100 mL, from about 0.2 mL to about 75 mL, from about 0.3 mL to about 50 mL, from about 0.4 mL to about 25 mL, from about 0.5 mL to about 20 mL, from about 0.6 mL to about 15 mL, from about 0.7 mL to about 10 mL, from about 0.8 mL to about 5 mL, from about 0.9 mL to about 2.5 mL, or from about 1 mL to about 2 mL. In other embodiments, the amount of formulation applied is about 0.1 mL, about 0.2 mL, about 0.3 mL, about 0.4 mL, about 0.5 mL, about 0.6 mL, about 0.7 mL, about 0.8 mL, about 0.9 mL, about 1.0 mL, about 1.5 mL, about 2 mL, about 2.5 mL, about 5 mL, or about 10 mL.

In another embodiment, the disclosed formulations are prepared as a lotion, a cream or a gel. The formulations can be packaged in a suitable container to suit its viscosity and intended use by the subject. In one aspect of this embodiment, the lotion or cream or gel can be packaged in a bottle or a roll-ball applicator, or a propellant-driven aerosol device or a container fitted with a pump suitable for finger operation. For example, when the formulation is a cream, it can be stored in a non-deformable bottle or in a squeeze container, such as a tube or a lidded jar. The present disclosure therefore, further provides a closed container the formulations disclosed herein.

As demonstrated in the Examples below, the present disclosure further provides methods for preparing formulations according to the present disclosure. Other methods, sources of materials, equipment, and procedures for preparing and packaging the formulations of the present disclosure will be apparent to those of ordinary skill in light of the present disclosure.

### EXAMPLES

### Example 1

### Formulation Protocol

This example provides an illustrative method of preparing a formulation of the disclosure. In particular, the individual components are prepared in seven separate “stages,” as noted below, and the individual stages are combined and mixed in the order and under the conditions specified in Example 2, below.

The A stage mixture: dissolve 0.20 g versene disodium salt crystals (EDTA, disodium salt) Surfachem (Leeds, UK) in 43.05 g deionized water to provide Stage A mixture

The B stage mixture: combine 5.00 g cetearyl alcohol (Cognis, Ludwigshaven, Germany), with 4 g castor (Ricinus communis seed) oil #1 USP grade, 2.00 g cetearyl alcohol/ceteareth 20 mixture (Procol CS-20 D, Protameen Chemicals Inc., Totowa N.J.), 5.00 g glyceryl stearate self-emulsifying glyceryl monostearate (Imwitor 960K, Cremor, Hamburg, Germany), 1.50 g D,L-alpha tocopheryl acetate oil (96) (Vitablend, Wolvega, The Netherlands), 5.00 sweet almond oil refined, 2.00 g steartic acid (Surfachem), and 1.00 gm cetyl esters (Crodamol SS-PA-(RB), Croda, Edison NJ), to provide the Stage B mixture.

The C stage mixture: Combine 0.25 g aloe vera 200:1 powder with 3.00 g deionized water to provide the Stage C mixture.

The D stage mixture: Combine 3.5 g Mexican wild yam (Dioscorea villosa) root liquid extract, 3.5 g panax ginseng root liquid extract (1:3 50%), 3.5 g saw palmetto liquid extract (1:3 50%), 3.5 g Damiana aphrodisiaca liquid extract (1:3 50%), and 3.50 g Dong quai liquid extract (1:3 50%), and 2.00 g gotu kola liquid extract (1:3 50%), to provide the Stage D mixture.

The E stage mixture: Combine 4 g USP micronized progesterone with 1.00 g Biovert substrate solution, which solution comprises 45-55% glucose, 0.66-0.8% potassium iodide, and 0.42-0.52% sodium thiocyanate (Arch Chemicals Inc., Allendale N.J.), to provide the State E mixture.

The F stage mixture: Combine 3.25 g deionized water with 0.05 g enzyme mixture (1000-1800 units/ml lactoperoxidase and 1500-2750 units/ml glucose oxidase; Arch Chemicals Inc., Allendale N.J.), to provide the Stage F mixture.

The G stage component: 0.2 g sage oil (sage Salvia essential oil)

### Example 2

### Manufacturing Protocol

This example provides an illustrative method for manufacturing a composition of the disclosure employing the Stage A through Stage F mixtures prepared as in Example 1.

To the main vessel, add the Stage A mixture and begin heating with gentle stirring to70° C. to 75° C. To a second vessel, add the Stage B mixture and begin heating with gentle stirring to 70° C. to 75° C. When both the A and B Stage mixtures are at the correct temperature (70° C. to 75° C.), add the B Stage mixture to the A stage mixture and begin homogenizing. When fully mixed, switch off homogenizer and being cooling with gentle stirring.

Premix the Stage C mixture and the Stage D mixture (Example 1). When the temperature in the main vessel is 35° C. to 40° C., add both the Stage C mixture and the Stage D mixture, stirring between each addition. Continue stirring and cooling until the product in the main vessel is below 33° C. Then very carefully add the Stage E mixture, and continue stirring. Prepare the Stage F mixture (Example 1) and carefully add the Stage F mixture to the main vessel. Finally, add the Stage G component and homogenize for 10 minutes, while cooling to ambient temperature.

The final product can then be distributed to appropriately-sized/appropriately-shaped containers for labeling and distribution.

As used throughout, ranges are used as shorthand for describing each and every value that is within the range. Any value within the range can be selected as the terminus of the range. In addition, all references cited herein are hereby incorporated by referenced in their entireties. In the event of a conflict in a definition in the present disclosure and that of a cited reference, the present disclosure controls.

Unless otherwise specified, all percentages and amounts expressed herein and elsewhere in the specification should be understood to refer to percentages by weight.

What is claimed is:

1. A formulation comprising:
water, an effective amount of a steroidal agent, an effective amount of a plant extract, an effective amount of a viscosity-adjusting agent, an effective amount of an anti-oxidant, and an effective amount of a chelator;
wherein the formulation is a topical composition for application to the skin.

2. The formulation of claim 1, wherein the steroidal agent is micronized progesterone.

3. The formulation of claim 1, wherein the viscosity-adjusting agent is an emollient, an emulsifier, a surfactant, or a thickener.

4. The formulation of claim 1, wherein the plant extract is aloe vera gel, Mexican wild yam root extract, sweet almond oil, panax ginseng root extract, saw palmetto extract, damiana aphrodisiaca extract, gotu kola extract, sage oil, dong quai root extract, castor oil, or combinations thereof.

5. The formulation of claim 1, wherein the viscosity-adjusting agent is cetearyl alcohol, ceteareth 20, glyceryl stearate, stearic acid, or cetyl esters.

6. The formulation of claim 1, wherein the chelator is ethylenediaminetetraacetic acid or a salt thereof.

7. The formulation of claim 1, wherein the antioxidant is D,L-tocopherol or a glucose/glucose oxidase/lactoperoxidase mixture.

8. The formulation of claim 2, wherein the formulation comprises, by weight, from about 2% to about 8% micronized progesterone.

9. The formulation of claim 4, wherein the plant extract is aloe vera 200:1 powder formulated within a range of from about 0.05% to about 0.5% by weight of the formulation, Mexican wild yam root extract formulated within a range of from about 1.5% to about 11% by weight of the formulation, sweet almond oil formulated within a range of from about 2.5% to about 7.5% by weight of the formulation, panax ginseng root extract formulated within a range of from about 1.5% to about 7% by weight of the formulation, saw palmetto extract formulated within a range of from about 1.5% to about 4.5% by weight of the formulation, damiana aphrodisiaca liquid extract (1:3 50%) formulated within a range of from about 0.5% to about 5% by weight of the formulation, gotu kola liquid extract (1:3 50%) formulated within a range of from about 0.5% to about 3.5% by weight of the formulation, sage oil formulated within a range of from about 0.05% to about 1.5% by weight of the formulation, dong quai root extract formulated within a range of from about 1.5% to about 5.5% by weight of the formulation, castor oil formulated within a range of from about 3% to about 6% by weight of the formulation, or a combination of two or more thereof.

10. The formulation of claim 5, wherein the viscosity-adjusting agent is cetearyl alcohol formulated within a range of from about 1% to about 8% by weight of the formulation, ceteareth 20 formulated within a range of from about 1% to about 8% by weight of the formulation, glyceryl stearate formulated within a range of from about 2.5% to about 7.5% by weight of the formulation, stearic acid formulated within a range of from about 1% to about 3% by weight of the formulation, cetyl esters formulated within a range of from about 0.5% to about 1.5% by weight of the formulation, or a combination of two or more thereof.

11. The formulation of claim 6, wherein the chelator is disodium ethylenediaminetetraacetic acid, formulated within a range of from about 0.1% to about 0.3% by weight of the formulation.

12. The formulation of claim 7, wherein the antioxidant is D,L-tocopherol formulated within a range of from about 0.5% to about 2.5% by weight of the formulation, or the mixture comprising glucose/glucose oxidase/lactoperoxidase, the mixture formulated with a range of from about 0.5% to about 1.5% by weight of the formulation, or a combination thereof.

13. The formulation of claim 1, comprising from about 40% to about 60% water, by weight.

14. The formulation according to claim 1, comprising, by weight, about 4% micronized progesterone, about 3.5% Mexican wild yam root extract, about 2% cetearyl alcohol and ceteareth 20, about 5% glyceryl stearate, about 2% stearic acid, about 5% sweet almond oil, about 1.5% D,L-alpha tocopheryl acetate oil, and about 0.2% disodium ethylenediaminetetraacetic acid.

15. The formulation of claim 14, further comprising about 51% water and about 0.25% aloe vera 200:1 powder.

16. The formulation of claim 14, further comprising about 49% water, about 0.25% aloe vera 200:1 powder, about 3.5% panax ginseng root extract (1:3 50%), about 3.5% saw palmetto extract (1:3 50%), about 3.5% damiana aphrodisiaca liquid extract (1:3 50%), about 2% gotu kola liquid extract (1:3 50%), and about 0.2% sage extract (sage Salvia essential oil).

17. The formulation according to claim 1, comprising about 49.3% water, about 4% micronized progesterone, about 2% disodium ethylenediaminetetraacetic acid, about 5% cetearyl alcohol, about 4% castor oil, about 2% cetearyl alcohol and ceteareth 20, about 5% glyceryl monostearate, about1.5% D,L-alpha tocopheryl acetate oil (96%), about 5% sweet almond extract, about 2% stearic acid, about 1% cetyl esters, about 0.25% aloe vera 200:1 powder; about 3.5% Mexican wild yam root liquid extract, about 3.5% panax ginseng root liquid extract (1:3 50%), about 3.5% saw palmetto liquid extract (1:3 50%), about 3.5% damiana aphrodisiaca liquid extract (1:3 50%), about 3.5% dong quai root liquid extract (1:3 50%), about 2% gotu kola liquid extract (1:3 50%), about 1% substrate (about 50% glucose, about 0.73% potassium iodide, and about 0.47% sodium thiocyanate), about 0.05% enzyme (lactoperoxidase 1000-1800 units/ml and glucose oxidase 1500-2750 units/ml), and about 0.2% sage Salvia essential oil.

18. The formulation of claim 1, wherein the formulation is prepared as a cream.

19. A method of conditioning the skin of a subject in need thereof, the method comprising topically applying an effective amount of the formulation of claim 1 to the skin of the subject.

20. The method of claim 19, wherein the subject is a male human.
